(19) United States
(12) Patent Application Publication
(10) Pub. No.: US 2009/0036993 A1
(43) Pub. Date: Feb. 5, 2009

(54) Patellar implant

(76) Inventor: Robert Metzger, Wakarusa, IN (US)

Correspondence Address: HARNESS, DICKEY & PIERCE, P.L.C., P.O. BOX 828, BLOOMFIELD HILLS, MI, 48303, US

(21) Appl. No.: 10/829,995

(22) Filed: Apr. 22, 2004

(51) Int. Cl. A61F 2/38

(52) U.S. Cl. 623/20.19; 623/20.21

(57) ABSTRACT

A patellar implant for a knee joint prosthesis including a femoral component and a tibial assembly having a post. The patellar implant includes an anterior anchoring surface, and a posterior surface articulating with the femoral component. The posterior surface has an articulating portion and a non-articulating portion, and the non-articulating portion defines a depression that substantially prevents impingement of the patellar implant on the post during flexion.

In total knee prostheses in which the posterior cruciate ligament is damaged or sacrificed, the tibial component includes a central tibial post which articulates with a cam of the femoral component to provide the required stability during flexion. Since their early introduction, these posterior-stabilized (PS) knee prostheses have undergone many modifications to improve the range of motion, increase posterior stability, and prevent or decrease the chance of adverse occurrences such as, for example, flexion instability, and patellar subluxation and dislocation. Nevertheless, impingement of the tibial post against the patellar component in deep flexion can still occur in posterior stabilized total knee arthroplasty. Post impingement can cause wear of the post and the patellar component, and possible fatigue fracture of the post. Post impingement can also limit flexion, and interfere with the extensor mechanism.

There is, therefore, a need for improvements in posterior-stabilized knee implants to prevent post impingement.

### SUMMARY

The present teachings provide a patellar implant for a knee joint prosthesis including a femoral component and a tibial assembly having a post. The patellar implant includes an anterior anchoring surface, and a posterior surface articulating with the femoral component. The posterior surface has an articulating portion and a non-articulating portion, and the non-articulating portion defines a depression that substantially prevents impingement of the patellar implant on the post during flexion.

The present teachings provide a knee joint prosthesis that includes a tibial assembly with a tibial post having posterior and anterior surfaces, a femoral component operable for articulation with the tibial bearing, the femoral component including a trochlear groove, and a patellar implant having a posterior surface shaped to articulate with the trochlear groove substantially without impinging on the anterior surface of the post during flexion.

The present teachings also provide a method of reducing impingement of a patellar implant on an anterior surface of a tibial post of a knee joint prosthesis during flexion. The method includes identifying articulating and non-articulating portions of a posterior surface of the patellar implant relative to a femoral component of the knee prosthesis, and shaping the non-articulating portion of the posterior surface of the patellar implant to substantially prevent impingement of the patellar implant on the tibial post.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is an environmental sectional view of a posterior-stabilized knee prosthesis shown in extension according to the present teachings;

FIG. 2 is an anterior view of a posterior-stabilized knee prosthesis shown in extension according to the present teachings;

FIG. 3 is the posterior-stabilized knee prosthesis of FIG. 1 shown in deep flexion;

FIG. 4 is a sectional view of a patellar implant according to the present teachings; and

FIG. 5 is a posterior view of patellar implant according to the present teachings.

### DETAILED DESCRIPTION

The following description is merely exemplary in nature and is in no way intended to limit the invention, its application, or uses. In this regard, while the invention is described in detail below generally with respect to posterior-stabilized (PS) knee joint prosthesis, the invention is not so limited, but can be applied to other types of knee joint prostheses, including cruciate retaining (CR), fully constrained, or hinged prostheses, and to prostheses with fixed, mobile or floating tibial bearings, etc.

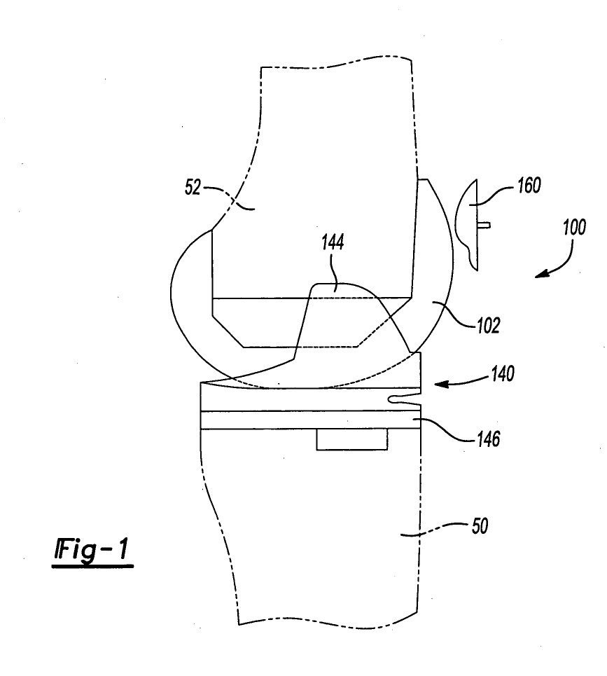

Referring to FIGS. 1-3, an exemplary posterior-stabilized (PS) knee joint prosthesis 100 according to the present teachings includes a femoral component 102, a tibial assembly 140 and a patellar implant 160. The knee joint prosthesis is secured to the tibia 50 and femur 52 of a resected knee joint. Although the knee joint prosthesis 100 is illustrated for a left knee joint, it will be appreciated that a similar prosthesis can be constructed for the right knee joint. Additionally, it will be understood that the present teachings are not limited to conventional posterior stabilized knee prostheses, but are also applicable to other knee prostheses that may have some degree of posterior constraint that may include a tibial post or equivalent structure. Moreover, although a fixed bearing knee joint prosthesis is illustrated, a mobile or floating bearing may also be employed.

The tibial assembly 140 can include a tibial bearing 142, a tibial post 144 and a tibial base 146 with an anchoring extension 147. Some or all of these components can be modular or integrally connected. For example, the tibial bearing 142 can be integral with the tibial post 144, but modularly coupled to the tibial base 146. Similarly, the tibial post 144 may be modularly or integrally. connected to the tibial bearing 142 or the tibial base 146. Or, the tibial bearing 142, the tibial post 144 and the tibial base 146 can be formed as one integral tibial assembly 140.

The post 144 has a posterior surface 150 and an anterior surface 148. The posterior surface 150 is operable to engage a central cam 104 of the femoral component 102 for at least some range of flexion. The central cam 104 extends between two condylar portions 111, 109 of the femoral component 102. The condylar portions 111, 109 include respective bearing surfaces 113, 115 for articulation with the tibial bearing 142. The shape, articulation and engagement of the femoral component 102, the tibial bearing 142, the tibial base 146, and the post 144, can be of any type known in the art, such as described, for example, in co-owned U.S. Pat. No. 6,579,283, No. 6,413,279, and No. 6,165,223, all of which are incorporated herein by reference. The tibial bearing 142, for example, can be of the type which is fixed relative to the tibial base 146, or of the floating type, such that the tibial bearing 142 can move relative to the tibial base 142 at least above a certain degree of flexion. Similarly, the tibial post 144 can be a monolithic component for a fixed tibial bearing 142. The tibial post 144 can be modular, including a portion fixed to the tibial base 146 and a portion that can be removed to alter the constraint provided by the joint knee implant.

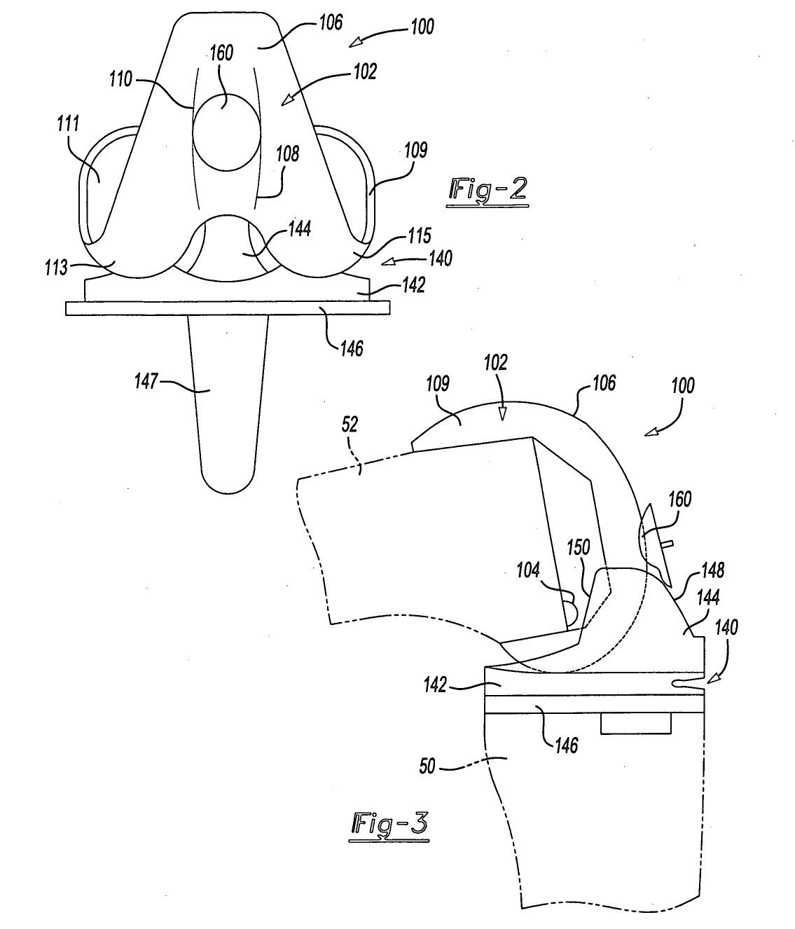

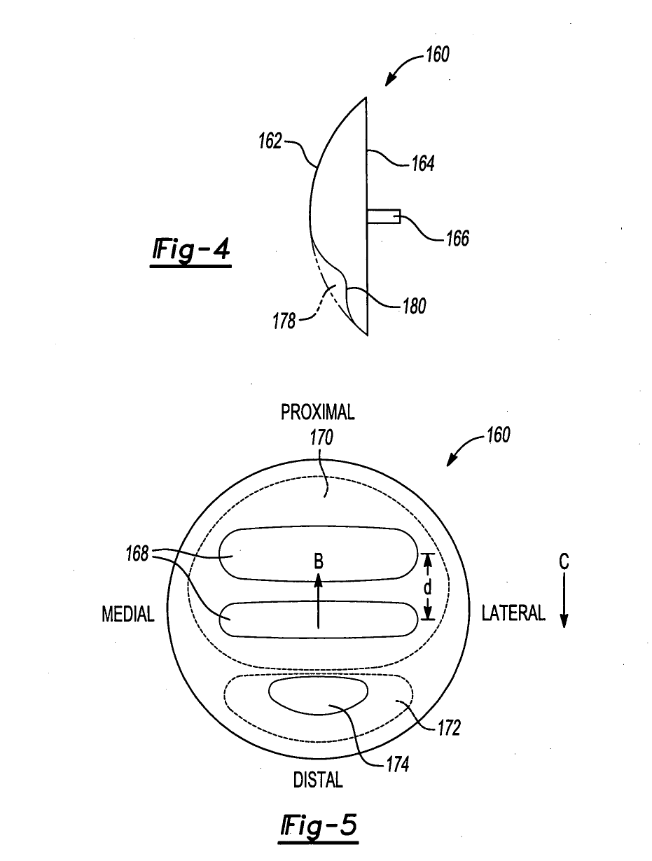

Referring to FIGS. 2, and 4, the anterior surface 106 of the femoral component 102 defines a trochlear groove 106 between a lateral ridge 108 and a medial ridge 110. The trochlear groove 106 is shaped for articulation with the patellar implant 160 during flexion. The patellar implant 160 has a posterior surface 162 and an anterior surface 164 from which one or more anchors 166 extend. The anchors 166 can be pegs, keels, posts, etc., and can include fins, threading or porous coating to improve anchoring. In extension or)° of flexion, the patellar implant 160 sits adjacent to the troclear groove 106. At about 15°-20° flexion, the patellar implant 160 engages the trochlear groove 106. The lateral ridge 108 functions as a buttress to prevent lateral translation of the patellar implant 160 during flexion.

The contact area 168 between the posterior surface 162 of the patellar implant 160 and the groove 106 increases with continued flexion and shifts distal (inferior) to proximal (superior) on the patellar implant 160 in the direction of the arrow “B”. The envelope of the successive positions of the contact area 168 define an articulating portion 170 and a non-articulating portion 172, which is located distally (inferiorly) relative to the articulating portion 170 on the posterior surface 162 of the patellar implant 160. During flexion, the patellar implant 160 moves distally (inferiorly) relative to the femoral component 102 by a distance “d” from full extension to full flexion in the direction of the arrow “C”. The distance d depends on the particular knee prosthesis system, but the distance d is about 7 cm for a natural knee joint. During deep (or high) flexion, as, for example, in stair-climbing, squatting, etc., the distal migration of the patellar implant 160 can cause the distal portion of the posterior surface 162 of the patellar implant 160 to impinge a portion of the anterior surface 148 of the tibial post 144, and commonly the superior/anterior corner of the tibial post 144. Impingement can occur for angles of flexion greater than about 120°, depending on the particular knee prosthesis system. Such occurrence can be avoided according to the present teachings for any flexion angle, including angles of flexion of 150° or greater, by removing material 178 from a non-essential distal portion 174 of the non-articulating portion 172 of the patellar implant 160 or otherwise shaping the non-articulating portion 172 to define a depression 180 on the non-articulating portion 172. The depression 180 can be concave, as illustrated in FIG. 4, although the depression 180 can have any shape or size that results from removing at least as much material 178 as needed from what could otherwise be a convex posterior surface 162 with symmetric proximal and distal portions. The depression 180 could be, for example, stepped, curved, polyhedral, continuous, discontinuous, etc., and combinations thereof. The depression 180 could be also shaped to conform to, without actually contacting for any degree of flexion, the anterior surface 148 of the tibial post 144.

Although the patellar implant 160 can be constructed by carving out material 178 from an existing prior art patellar implant that has a convex, domed posterior surface, as shown in phantom line in FIG. 4, the patellar implant 160 can be also constructed directly by molding polyethylene, such as compression molded polyethylene, to produce a posterior surface that has a convex articulating portion 170 transitioning to a concave non-articulating position. The posterior surface 162 of the patellar implant 160 can be shaped such that impingement is prevented for any degree of flexion permitted by the knee joint prosthesis, or, if desired, up to a predetermined degree of flexion. The patellar implant 160 can be also constructed from other biocompatible materials including metal, polymer, ceramic, composite, etc. Similarly, the tibia bearing 142, the tibial post 144 and the tibial base 146 can be constructed integrally or modularly from other biocompatible materials including metal, polymer, ceramic, composite, etc., or combination thereof.

It will be appreciated that the patellar implant 160 permits deep flexion for any angle of flexion, including angles of flexion greater than 150°, without post impingement and associated wear of the tibial post 144, thereby facilitating the design of the other components of the knee prosthesis to provide the required degree of stability and flexibility.

While particular embodiments have been described in the specification and illustrated in the drawings, it will be understood by those skilled in the art that various changes may be made and equivalents may be substituted for elements thereof without departing from the scope of the invention as defined in the claims. In addition, many modifications may be made to adapt a particular situation or material to the present teachings without departing from the essential scope thereof. Therefore, it is intended that the present teachings are not be limited to the particular embodiments illustrated by the drawings and described in the specification, but that the present teachings will include any embodiments falling within the foregoing description and the appended claims.

1. A knee joint prosthesis comprising:
a femoral component;
a tibial assembly having a post extending proximally from the tibial assembly toward the femoral component; and
a patellar implant comprising:
an anterior anchoring side;
a posterior side having medial, lateral, superior and inferior sides defined relative to the femoral component, the posterior side defining a three-dimensional posterior surface bounded by a circular circumference, the posterior surface substantially consisting of convex articulating proximal portion extending from the medial side to the lateral side and a concave distal non-articulating portion extending from the medial side to the lateral side, the distal non-articulating portion positioned directly inferiorly to the proximal articulating portion, the articulating proximal portion shaped to articulate in contact with the femoral component during flexion and the non-articulating distal portion shaped to substantially prevent impingement of the patellar implant on the post during any degree of flexion, the non-articulating portion not contacting the femoral component at any degree of flexion.

2. (canceled)

3. The prosthesis of claim 1, wherein the patellar implant is constructed from compression molded polyethylene.

4. The prosthesis of claim 1, wherein the patellar implant is constructed from biocompatible material selected from the group consisting of metal, polymer, ceramic, composite, and combinations thereof.

5. The prosthesis of claim 1, wherein flexion includes angles of flexion greater than 110°.

6-7. (canceled)

8. The prosthesis of claim 1, wherein the patellar implant is further comprising at least one anchor extending from the anterior anchoring side.

9. The prosthesis of claim 8, wherein the anchor is selected from the group consisting of peg, keel, and post.

10. A knee joint prosthesis comprising:
a tibial assembly including a tibial post extending superiorly relative to an articulating surface of the tibial assembly, the post having posterior and anterior surfaces;
a femoral component articulatable with a portion of the articulating surface of the tibial assembly and the post, the femoral component including a trochlear groove; and
a patellar implant having a posterior surface, the posterior surface consisting of a proximal convex portion shaped to articulate with the trochlear groove and a distal concave portion shaped to substantially avoid impinging on the post during any degree of flexion, the distal concave portion not contacting the trochlear groove at any degree of flexion.

11. The knee prosthesis of claim 10, wherein the tibial assembly further comprises a tibial bearing articulating with the femoral component.

12. The knee prosthesis of claim 11, further comprising a tibial base coupled to the tibial bearing.

13. The knee prosthesis of claim 12, wherein the tibial bearing is selected from the group consisting of fixed, mobile, and floating.

14. The knee prosthesis of claim 13, wherein the tibial post is fixed on the tibial base.

15. The knee prosthesis of claim 11, wherein the post is coupled to the tibial bearing.

16. The knee joint prosthesis of claim 12, wherein the tibial base is modularly connected to the tibial bearing.

17. The knee joint prosthesis of claim 11, wherein the tibial post is modularly connected to the tibial bearing.

18. The knee joint prosthesis of claim 11, further comprising at least one anchor extending from an anterior surface of the patellar implant.

19. The knee joint prosthesis of claim 18, wherein the anchor is selected from the group consisting of peg, keel, and post.

20. A method of reducing impingement of a patellar implant on a proximal tibial post of a posterior-stabilized knee joint prosthesis during flexion, the tibial post extending superiorly from a tibial component toward a femoral component of the knee joint prosthesis, the method comprising:
identifying articulating and non-articulating portions of a posterior surface of the patellar implant relative to a femoral component of the knee joint prosthesis;
forming the articulating portion as a portion of a convex dome; and
forming the non-articulating portion by replacing a portion of the convex dome with a concave portion;
positioning the patellar implant relative to a trochlear groove of the femoral component;
contacting the trochlear groove only with a portion of the convex dome during flexion; and
preventing impingement of the patellar implant on the proximal tibial post during any degree of flexion.

21. The method of claim 20, wherein replacing a portion of the convex dome with a concave portion comprises removing material from the convex dome.

22. The method of claim 20, wherein replacing a portion of the convex dome with a concave portion comprises molding the articulating and non-articulating portions.

23. The the prosthesis of claim 1, wherein the distal non-articulating portion is under a surface that would be defined by extending the convex articulating proximal portion distally toward the tibial assembly.
